(19) United States
(12) Patent Application Publication
(10) Pub. No.: US 2026/0248989 A1
(43) Pub. Date: Aug. 27, 2026

(54) SINGLE NEEDLE DIALYSIS SYSTEM AND METHODS

(71) Applicant: OUTSET MEDICAL, INC., San Jose, CA (US)

(72) Inventor: Dean HU, San Leandro, CA (US)

(21) Appl. No.: 18/874,742

(22) PCT Filed: Jun. 14, 2023
(86) PCT No.: PCT/US2023/068459

§ 371 Date: Dec. 13, 2024

Related U.S. Application Data
(60) Provisional application No. 63/351,928, filed on Jun. 14, 2022.

(51) Int. Cl. A61M 1/16; A61M 1/30; A61M 1/36

(52) U.S. Cl. CPC A61M 1/1656; A61M 1/302; A61M 1/3622

(57) ABSTRACT

Dialysis systems and methods are described which can include a number of features. The dialysis systems described can be to provide dialysis therapy to a patient in the comfort of their own home. The dialysis system can be configured to prepare purified water from a tap water source in real-time that is used for creating a dialysate solution. The dialysis systems described also include features that make it easy for a patient to self-administer therapy.

### CROSS REFERENCE TO RELATED APPLICATIONS

This application claims the benefit of priority of U.S. Provisional Application No. 63/351,928, filed Jun. 14, 2022, herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to dialysis systems. More specifically, this disclosure relates to systems and methods for creating dialysate in real-time during dialysis treatment.

### BACKGROUND

There are, at present, hundreds of thousands of patients in the United States with end-stage renal disease. Most of those require dialysis to survive. Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Many dialysis systems on the market require significant input and attention from technicians prior to, during, and after the dialysis therapy. Before therapy, the technicians are often required to manually install patient blood tubing sets onto the dialysis system, connect the tubing sets to the patient, and to the dialyzer, and manually prime the tubing sets to remove air from the tubing set before therapy. During therapy, the technicians are typically required to monitor venous pressure and fluid levels, and administer boluses of saline and/or heparin to the patient. After therapy, the technicians are often required to return blood in the tubing set to the patient and drain the dialysis system. The inefficiencies of most dialysis systems and the need for significant technician involvement in the process make it even more difficult for patients to receive dialysis therapy away from large treatment centers.

Given the demanding nature of in-center dialysis, many patients have turned to home dialysis as an option. Home dialysis provides the patient with scheduling flexibility as it permits the patient to choose treatment times to fit other activities, such as going to work or caring for a family member. Unfortunately, current dialysis systems are generally unsuitable for use in a patient's home. One reason for this is that current systems are too large and bulky to fit within a typical home. Current dialysis systems are also energy-inefficient in that they use large amounts of energy to heat large amounts of water for proper use. Although some home dialysis systems are available, they generally are difficult to set up and use. As a result, most dialysis treatments for chronic patients are performed at dialysis centers.

Hemodialysis is also performed in the acute hospital setting, either for current dialysis patients who have been hospitalized, or for patients suffering from acute kidney injury. In these care settings, typically a hospital room, water of sufficient purity to create dialysate is not readily available. Therefore, hemodialysis machines in the acute setting rely on large quantities of pre-mixed dialysate, which are typically provided in large bags and are cumbersome for staff to handle. Alternatively, hemodialysis machines may be connected to a portable RO (reverse osmosis) machine, or other similar water purification device. This introduces another independent piece of equipment that must be managed, transported and disinfected.

Dialysis machines are used in a variety of settings, including hospital rooms, dedicated clinics and patient homes. In some settings, minimal mobility requirements are needed, such as in the home or the clinic setting. In other settings, such as hospital rooms, mobility could be very important. The machine may need to be transported across long distances, hallways, or even exterior surfaces going from one building to another. Additionally, within a hospital room, space is at a premium, and high maneuverability e.g., ability to spin about its own axis, is desirable. However, mobility solutions that are optimized for one setting may not work well in other settings for size, footprint or cost reasons. Therefore, there is a need for a modular approach where a single dialysis machine could have an option of mobility solutions, and preferably where the installation of that modular mobility solution is minimally burdensome.

Pre-configured dialysis machines are those which have onboard water purification hardware, such as a reverse osmosis system. These systems often have a number of water filters, such as sediment, carbon and ultrafilters that purify the water that is later used to create dialysate. The quality of the incoming water has a significant impact on the life of many of these filters. Factors such as sediment content, chlorine/chloramine concentration, hardness, pH, alkalinity and temperature can shorten the lifespan of filters and/or impact the quality of the water after it is filtered. Due to highly varied nature of the incoming water, different options for treating the water would be desired. It could be conceivable to produce single a water treatment system that could handle a wide range of input variables, although doing so may be prohibitive from a size, weight, cost or maintenance standpoint. Therefore, there is a need for a modular approach for water prefiltration, and preferably one where maintenance such as changing filters is minimally burdensome.

Single needle dialysis has typically been enabled by utilizing two blood pumps and an accumulator reservoir located between them. The upstream blood pump will withdraw blood and deliver into the accumulator, while the downstream pump is inactive. Then, the upstream pump will stop pumping, and the downstream pump will pump blood from the accumulator, through the dialyzer, and into the patient. This requires multiple blood pumps which is an additional cost.

### SUMMARY

A method of providing dialysis is provided, comprising: inserting a single needle of a dialysis system into a patient; activating an inflow phase of the dialysis system with a single blood pump that includes withdrawing blood from the patient through the single needle into a blood circuit of the dialysis system and into an accumulator reservoir of the blood circuit; and activating an outflow phase of the dialysis system with the single blood pump that includes moving blood from the accumulator reservoir through a dialyzer of the dialysis system and back into the patient through the single needle.

In one implementation, the inflow phase further comprises: closing a venous valve positioned on a venous line of the blood circuit; opening an arterial valve positioned on an arterial line of the blood circuit; opening a first valve positioned on a first fluid line that fluidly couples the accumulator reservoir to the blood circuit at a location downstream of the single blood pump; and closing a second valve positioned on a second fluid line that fluidly couples the accumulator reservoir to the blood circuit at a location upstream of the single blood pump.

In one aspect, the inflow phase comprises running the single blood pump in a forward direction.

In another aspect, the outflow phase further comprises: opening a venous valve positioned on a venous line of the blood circuit; closing an arterial valve positioned on an arterial line of the blood circuit; closing a first valve positioned on a first fluid line that fluidly couples the accumulator reservoir to the blood circuit at a location downstream of the single blood pump; and opening a second valve positioned on a second fluid line that fluidly couples the accumulator reservoir to the blood circuit at a location upstream of the single blood pump.

In one aspect, the outflow phase comprises running the single blood pump in a forward direction.

In some aspects, the inflow phase comprises filling the accumulator reservoir with blood.

In one aspect, the inflow phase comprises operating the single blood pump in a forward direction for a preset period of time.

In another aspect, the method includes syncing an ultrafiltration flow of dialysate through the dialyzer with the outflow phase.

In one aspect, the single blood pump operates at a substantially similar flow rate during the inflow phase and the outflow phase.

In some aspects, the single blood pump operates at a first flow rate during the inflow phase and a second flow rate during the outflow phase.

In one aspect, the first flow rate is faster than the second flow rate.

In some aspects, the first flow rate is slower than the second flow rate.

A dialysis system is provided, comprising: a blood circuit comprising an arterial line, a venous line, and a single needle connected to the arterial line and venous line; a blood pump configured to interact with the blood circuit to move a flow of blood through the blood circuit; a dialyzer fluidly coupled to the blood circuit; an accumulator reservoir fluidly connected to the blood circuit with a first line and a second line, the first line being coupled to the blood circuit downstream of the blood pump and the second line being coupled to the blood circuit upstream of the blood pump; a venous valve positioned on the venous line; an arterial valve positioned on the arterial line; a first valve positioned on the first line; a second valve positioned on the second line; an electronic controller operatively coupled to the blood pump, the venous valve, the arterial valve, the first valve, and the second valve, wherein the electronic controller is configured to: activate an inflow phase that comprises closing the venous valve and the second valve, opening the arterial valve and the first valve, and operating the blood pump in a forward direction to withdraw blood from the patient through the single needle into the blood circuit and into the accumulator reservoir via the first line; and activate an outflow phase that comprises opening the venous valve and the second valve, closing the arterial valve and the first valve, and operating the blood pump in the forward direction to move blood from the accumulator reservoir into the blood circuit via the second line and through the dialyzer and back into the patient through the single needle.

In one aspect, the inflow phase is stopped when the accumulator reservoir is filled with blood.

In another aspect, the inflow phase is stopped when the blood pump is operated in a forward direction for a preset period of time.

In some aspects, the electronic controller is further configured to sync an ultrafiltration flow of dialysate through the dialyzer with the outflow phase.

In one aspect, the blood pump operates at a substantially similar flow rate during the inflow phase and the outflow phase.

In some aspects, the blood pump operates at a first flow rate during the inflow phase and a second flow rate during the outflow phase.

In one aspect, the first flow rate is faster than the second flow rate.

In another aspect, the first flow rate is slower than the second flow rate.

A method of retrofitting a dual-needle dialysis system to operate as a single-needle dialysis system, comprising: forming a Y-junction between a venous line and an arterial line of a blood circuit of the dual-needle dialysis system; replacing first and second needles of the dual-needle dialysis system with a single needle at the Y-junction; replacing a saline source of the dual-needle dialysis system with an accumulator reservoir; inserting the single needle into a patient; activating an inflow phase of the dialysis system that includes withdrawing blood from the patient through the single needle into the blood circuit of the dialysis system and into the accumulator reservoir of the blood circuit; and activating an outflow phase of the dialysis system that includes moving blood from the accumulator reservoir through a dialyzer of the dialysis system and back into the patient through the single needle.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one embodiment of a dialysis system.

FIG. 2 illustrates one embodiment of a water purification system of the dialysis system.

FIG. 3 illustrates one embodiment of a dialysis delivery system of the dialysis system.

FIG. 4 illustrates one embodiment of a dual-needle dialysis delivery system.

FIGS. 5A-5C illustrate a single-needle dialysis delivery system and methods of use.

### DETAILED DESCRIPTION

This disclosure describes systems, devices, and methods related to dialysis therapy, including a dialysis system that is simple to use and includes automated features that eliminate or reduce the need for technician involvement during dialysis therapy. In some embodiments, the dialysis system can be a home dialysis system. Embodiments of the dialysis system can include various features that automate and improve the performance, efficiency, and safety of dialysis therapy.

In some embodiments, a dialysis system is described that can provide acute and chronic dialysis therapy to users. The system can include a water purification system configured to prepare water for use in dialysis therapy in real-time using available water sources, and a dialysis delivery system configured to prepare the dialysate for dialysis therapy. The dialysis system can include a disposable cartridge and tubing set for connecting to the user during dialysis therapy to retrieve and deliver blood from the user.

This disclosure provides systems and methods configured to deliver single needle dialysis using the same cartridge blood set and actuation hardware configuration intended to deliver dialysis using two needles. In some embodiments, a single needle cartridge blood set may be used in place of the one used for dual needle systems. Single needle dialysis is advantageous over double needle dialysis for two reasons; first, cannulation with a single needle rather than two is easier and less painful; and second, double needle dialysis poses the serious risk of exsanguination if the return line (venous) needle becomes dislodged, while the withdrawal line (arterial) remains connected. As the single needle serves as both the withdrawal and return line, if it becomes dislodged, no exsanguination threat is posed to the patient. This disclosure implements a staged flow approach to single needle dialysis, where the flow will alternate between withdrawing and returning blood, rather than a continuous flow scheme, where the single needle may have two lumens that withdraw and return blood simultaneously and continuously. It should be noted that the effective blood flow rate is halved in this configuration, which limits the ability to quickly complete a treatment and deliver high per-time clearances. However, treating for longer times has benefits, including a lower rate of fluid removal to remove the same target fluid volume, which can avoid blood volume depletion and intradialytic hypotensive events. Lower flows and longer treatment times are a natural fit with nocturnal hemodialysis, which takes advantage of time the patient is asleep and comes at low lifestyle opportunity cost, unlike daytime hemodialysis. Because the patient is not conscious and unable to react to most stimulus during nocturnal hemodialysis, the risk of venous needle dislodgment is higher, and a means to completely avoid this risk, such as single needle dialysis, is of high value.

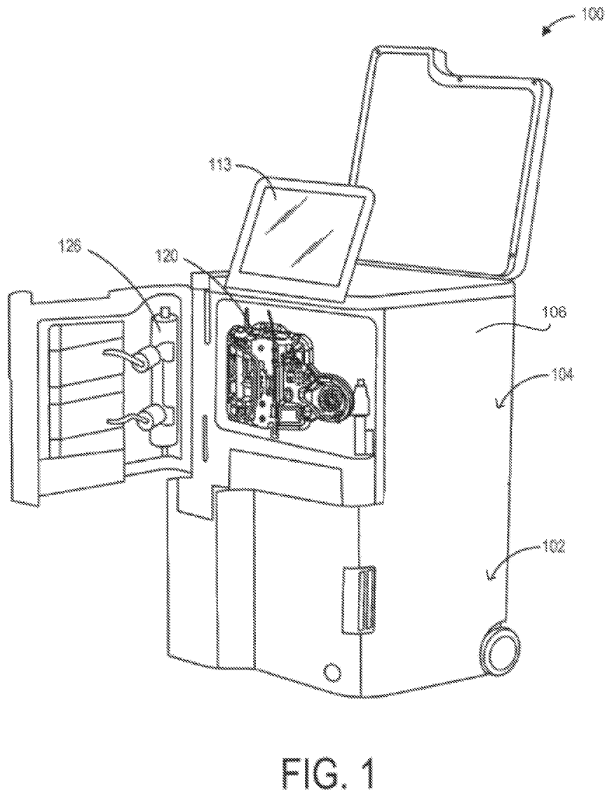

FIG. 1 illustrates one embodiment of a dialysis system 100 configured to provide dialysis treatment to a user in either a clinical or non-clinical setting, such as the user's home. The dialysis system 100 can comprise a water purification system 102 and a dialysis delivery system 104 disposed within a housing 106. The water purification system 102 can be configured to purify a water source in real-time for dialysis therapy. For example, the water purification system can be connected to a residential water source (e.g., tap water) and prepare purified water in real-time. The pasteurized water can then be used for dialysis therapy (e.g., with the dialysis delivery system) without the need to heat and cool large batched quantities of water typically associated with water purification methodologies.

Dialysis system 100 can also include a cartridge 120 which can be removably coupled to the housing 106 of the system. The cartridge can include a patient tubing set attached to an organizer. The cartridge and tubing set, which can be sterile, disposable, one-time use components, are configured to connect to the dialysis system prior to therapy. This connection correctly aligns corresponding components between the cartridge, tubing set, and dialysis system prior to dialysis therapy. For example, the tubing set is automatically associated with one or more pumps (e.g., peristaltic pumps), clamps and sensors for drawing and pumping the user's blood through the tubing set when the cartridge is coupled to the dialysis system. The tubing set can also be associated with a saline source of the dialysis system for automated priming and air removal prior to therapy. In some embodiments, the cartridge and tubing set can be connected to a dialyzer 126 of the dialysis system. In other embodiments, the cartridge and tubing set can include a built-in dialyzer that is pre-attached to the tubing set. A user or patient can interact with the dialysis system via a user interface 113 including a display.

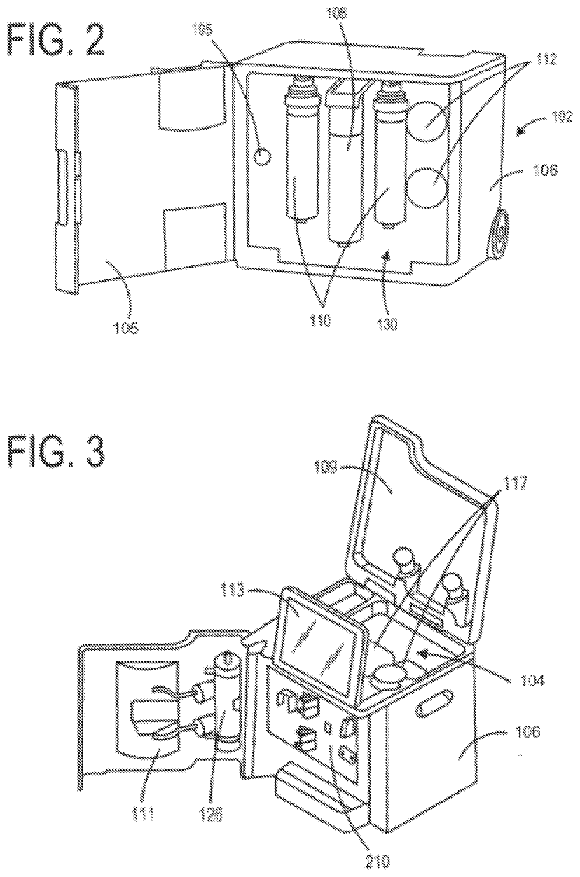

FIGS. 2-3 illustrate the water purification system 102 and the dialysis delivery system 104, respectively, of one embodiment of the dialysis system 100. The two systems are illustrated and described separately for ease of explanation, but it should be understood that both systems can be included in a single housing 106 of the dialysis system. FIG. 2 illustrates one embodiment of the water purification system 102 contained within housing 106 that can include a front door 105 (shown in the open position). The front door 105 can provide access to features associated with the water purification system such as one or more filters, including sediment filter(s) 108, carbon filter(s) 110, and reverse osmosis (RO) filter(s) 112. The filters can be configured to assist in purifying water from a water source (such as tap water) in fluid communication with the water purification system 102. The water purification system can further include heating and cooling elements, including heat exchangers, configured to pasteurize and control fluid temperatures in the system, as will be described in more detail below. The system can optionally include a chlorine sample port 195 to provide samples of the fluid for measuring chlorine content.

In FIG. 3, the dialysis delivery system 104 contained within housing 106 can include an upper lid 109 and front door 111, both shown in the open position. The upper lid 109 can open to allow access to various features of the dialysis system, such as user interface 113 (e.g., a computing device including an electronic controller and a display such as a touch screen) and dialysate containers 117. Front door 111 can open and close to allow access to front panel 210, which can include a variety of features configured to interact with cartridge 120 and its associated tubing set, including alignment and attachment features configured to couple the cartridge 120 to the dialysis system 100. Dialyzer 126 can be mounted in front door 111 or on the front panel, and can include lines or ports connecting the dialyzer to the prepared dialysate, dialysate concentrate, liquid concentrate, etc., as well as to the tubing set of the cartridge. In one implementation, described in more detail below, the dialysate machine and/or dialyzer can include lines or ports configured to connect to a disposable reservoir that includes a plurality of powdered and/or liquid compartments for dialysate preparation and delivery.

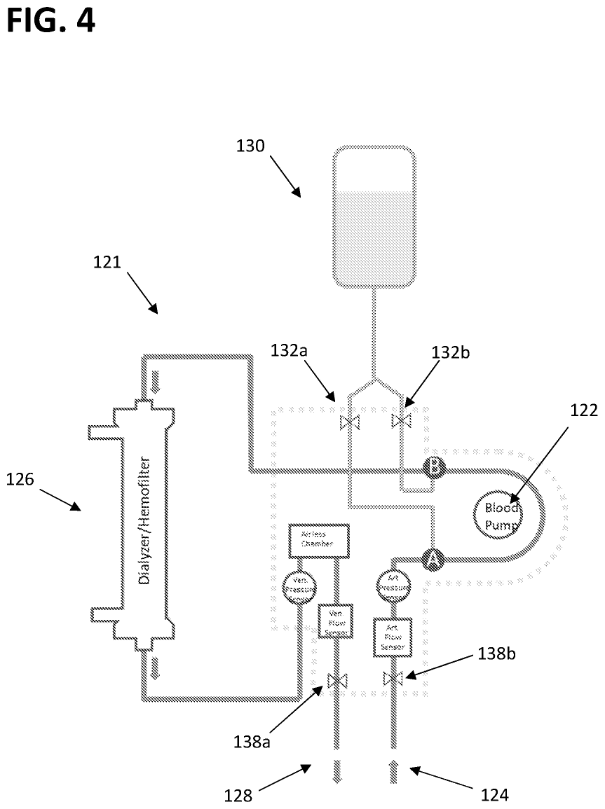

FIG. 4 illustrates a schematic of a typical double needle dialysis system. As shown, the system can include a dialyzer 126 fluidly coupled to a patient tubing set 121. A single blood pump 122 is configured to interact with the tubing set to move fluid (e.g., blood) through the tubing set. The tubing set can further include an airless or venous drip chamber to remove air from the tubing set. The tubing set can include an arterial access point 124 (e.g., arterial needle) and a venous access point 128 (e.g., venous needle). The venous and arterial access points can be selectively clamped with venous and arterial pinch valves 138a and 138b, respectively. As shown, the arterial needle receives blood in from the patient and the venous needle returns blood to the patient. The tubing set can include various sensors as shown (e.g., atrial and venous pressure and flow sensors). As is also shown in FIG. 4, a saline source 130 is connected to the blood flow path/patient tubing set with a bifurcated line with two independently controlled machine-actuated valves 132a and 132b. One junction, via valve 132a, is immediately upstream of the blood pump (A), and the other junction, via valve 132b, is immediately downstream of the blood pump (B). In practice, this allows the system to deliver saline both forwards and backwards into the blood flow by setting the input of the blood pump to the saline bag regardless if it is turning clockwise or counterclockwise. Saline from the bag is used for priming the cartridge, administering during treatment to relieve hypotensive symptoms or mitigate blood set clotting, and return blood. One or more processors or electronic controllers can be electrically coupled to the dialysis system, including but not limited to the blood pump 122 and valves 132a, 132b, 138a, and 138b, to control operation of the system during priming and therapy. While the discussion below describes electronic or automatic operation of the valves, such as with a controller or microprocessor, it should be understood that the valves could be manually actuated to achieve the same function. During therapy, the blood flows through the tubing set in the direction of the arrows when the blood pump operates in the forward (e.g., counter-clockwise) direction.

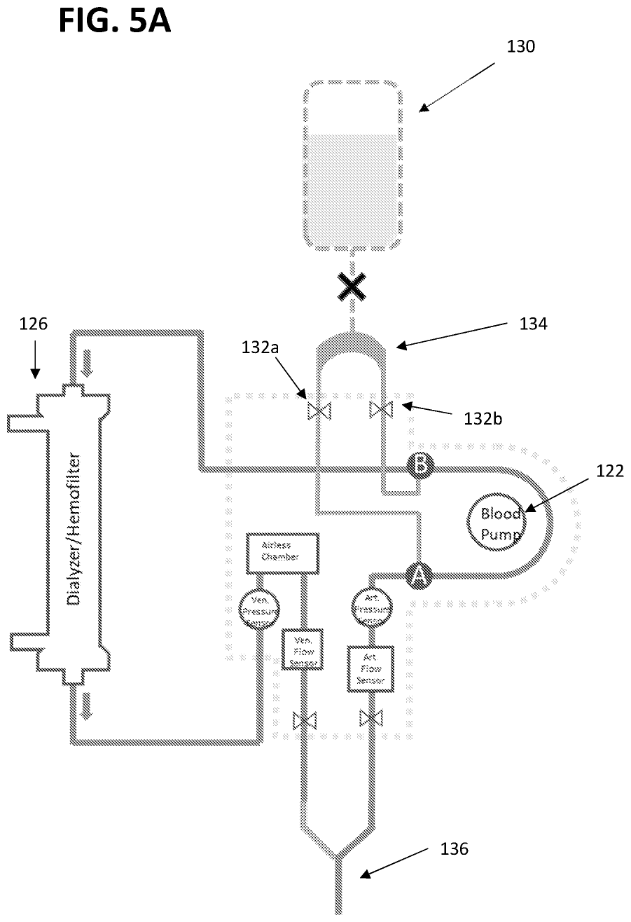

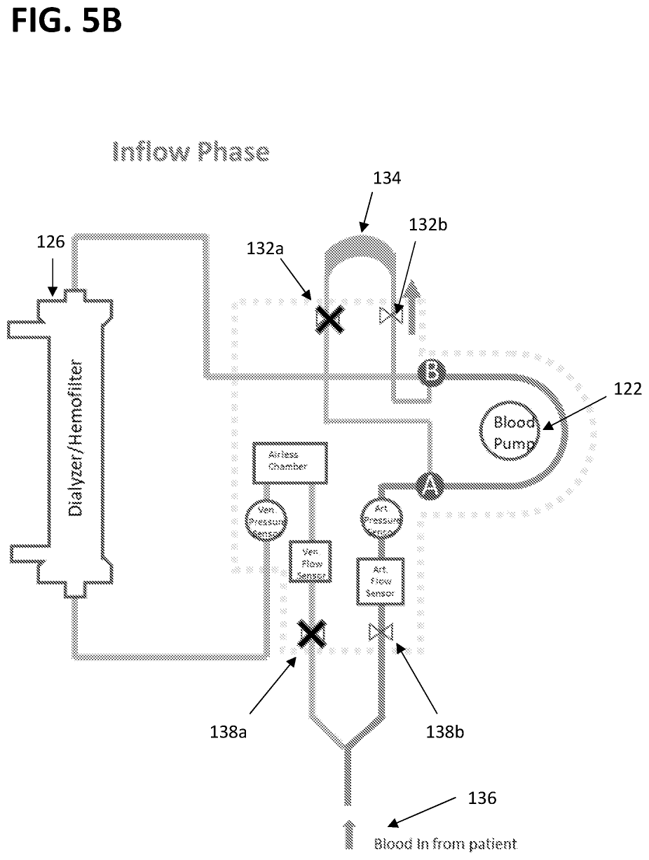

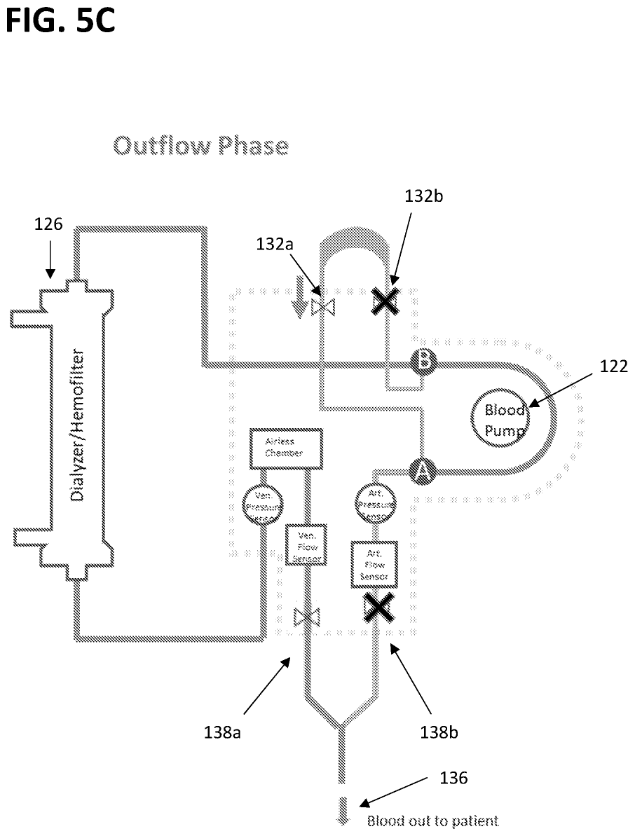

FIG. 5A is an embodiment of a single needle dialysis system. In some embodiments, a double needle dialysis system as described above in FIG. 4 can be retrofit or modified to work as a single needle system. In one example, the primary physical structure is identical to the flow path as illustrated and described above, with the addition of an accumulation reservoir 134 to replace the saline source 130 and a single needle 136 for removing blood from the patient and returning blood to the patient. However, the flow paths are identical between the single and double needle systems, with two lines coming from the accumulation reservoir attached to the same points (A) and (B) upstream and downstream of the blood pump via independently controlled machine-actuated valves 132a and 132b. This implementation assumes that the saline bag is no longer needed (at least from the functions of delivering boluses or flushes during treatment). However, in one example, a saline source could be attached to the accumulation reservoir, and a manual (or machine-actuated clamp) can be configured to isolate the saline source from the accumulation reservoir until needed (e.g., for blood return).

In some embodiments, the accumulation reservoir 134 could have the characteristics of being 1) flexible (high compliance) and 2) structured in such a way that blood flows through its entire volume. In this example, there is no area within the accumulation reservoir where blood can stagnate. In some embodiments, the accumulation reservoir could be formed by welding two flexible sheets together, with an inlet and outlet at opposite ends, with the flow path gradually widening from the inlet and then narrowing to the outlet.

To enable single needle dialysis with the system of FIG. 5A, an outflow phase (withdrawing blood from the patient) and an inflow phase (returning blood to the patient) are provided, as the system can no longer withdraw blood and return blood simultaneously as with a double needle system. In one embodiment, to make the single needle connection, the arterial and venous lines can be connected together, forming the two legs of a Y connection, with the head of the Y connecting to the single needle access.

FIG. 5B illustrates an inflow phase of the single needle dialysis system. During the inflow phase, the venous pinch valve 138a can be controlled to be closed, and valve 132b, which is between the downstream junction (B) and the accumulator chamber, can be controlled to be opened. The arterial pinch valve 138b can be controlled to be opened, while the valve 132a, which is between the upstream junction (A) and the accumulator chamber, can be controlled to be closed. The blood pump 122 can be activated, and as the blood pump turns in the forward (e.g., counter-clockwise) direction, blood is pumped from the patient through the single needle 136 into the tubing set, through open valve 138b, past upstream junction (A) into the accumulator chamber 134 via downstream junction (B), until the accumulator chamber 134 is filled, or until a predetermined volume has been pumped or time has passed.

Next, referring to FIG. 5C, during the outflow phase, the positions of all four valves are flipped. For example, the arterial pinch valve 138b is controlled to be closed and the venous pinch valve 138a is controlled to be opened, while the pre-pump valve 132a is controlled to be open and the post-pump valve 132b is controlled to be closed. As the blood pump 122 turns in the forward (e.g., counter-clockwise) direction in this configuration, blood is moved out of the accumulation reservoir 134 back into the tubing set at upstream junction (A), past downstream junction (B), through the dialyzer 126 and then back to the patient via the single needle 136.

This sequence repeats for the duration of treatment. The blood pump may continue to turn at a set speed through both phases, or the blood pump can be set to different flow rates for the inflow/outflow phases, although the total volume delivered by both phases should match.

The interaction of the accumulation reservoir with the fluid removal function of the dialysis machine is also considered. During standard dialysis, the dialysate flow of the dialyzer is intentionally unbalanced such that the volume of dialysate into the dialyzer is smaller than the volume of dialysate out of the dialyzer, with the extra fluid coming from the blood side of the fluid circuit. During the outflow phase, this is no different than standard dialysis, but during the inflow phase, the blood side of the dialyzer is effectively a sealed volume. Though there is some compliance in the tubing that would allow fluid to be removed from the blood during the inflow phase, it might not be ideal to do so, as some negative pressure may build up. Once the segment shifts to the outflow phase, this should be relieved, as the dialyzer blood side now fluidically communicates with the patient. This cyclic de-pressurization and relief could pose issues; therefore, in one embodiment the system is configured to sync the ultrafiltration flow with just the outflow phase, or to ensure that the phases are of short enough period that any pressurization effects from ultrafiltration are minimized.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed:

1. A method of providing dialysis, comprising:
inserting a single needle of a dialysis system into a patient;
activating an inflow phase of the dialysis system with a single blood pump that includes withdrawing blood from the patient through the single needle into a blood circuit of the dialysis system and into an accumulator reservoir of the blood circuit; and
activating an outflow phase of the dialysis system with the single blood pump that includes moving blood from the accumulator reservoir through a dialyzer of the dialysis system and back into the patient through the single needle.

2. The method of claim 1, wherein the inflow phase further comprises:
closing a venous valve positioned on a venous line of the blood circuit;
opening an arterial valve positioned on an arterial line of the blood circuit;
opening a first valve positioned on a first fluid line that fluidly couples the accumulator reservoir to the blood circuit at a location downstream of the single blood pump; and
closing a second valve positioned on a second fluid line that fluidly couples the accumulator reservoir to the blood circuit at a location upstream of the single blood pump.

3. The method of claim 2, wherein the inflow phase comprises running the single blood pump in a forward direction.

4. The method of claim 1, wherein the outflow phase further comprises:
opening a venous valve positioned on a venous line of the blood circuit;
closing an arterial valve positioned on an arterial line of the blood circuit;
closing a first valve positioned on a first fluid line that fluidly couples the accumulator reservoir to the blood circuit at a location downstream of the single blood pump; and
opening a second valve positioned on a second fluid line that fluidly couples the accumulator reservoir to the blood circuit at a location upstream of the single blood pump.

5. The method of claim 4, wherein the outflow phase comprises running the single blood pump in a forward direction.

6. The method of claim 1, wherein the inflow phase comprises filling the accumulator reservoir with blood.

7. The method of claim 1, wherein the inflow phase comprises operating the single blood pump in a forward direction for a preset period of time.

8. The method of claim 1, further comprising syncing an ultrafiltration flow of dialysate through the dialyzer with the outflow phase.

9. The method of claim 1, wherein the single blood pump operates at a substantially similar flow rate during the inflow phase and the outflow phase.

10. The method of claim 1, wherein the single blood pump operates at a first flow rate during the inflow phase and a second flow rate during the outflow phase.

11. The method of claim 10, wherein the first flow rate is faster than the second flow rate.

12. The method of claim 10, wherein the first flow rate is slower than the second flow rate.

13. A dialysis system, comprising:
a blood circuit comprising an arterial line, a venous line, and a single needle connected to the arterial line and venous line;
a blood pump configured to interact with the blood circuit to move a flow of blood through the blood circuit;
a dialyzer fluidly coupled to the blood circuit;
an accumulator reservoir fluidly connected to the blood circuit with a first line and a second line, the first line being coupled to the blood circuit downstream of the blood pump and the second line being coupled to the blood circuit upstream of the blood pump;
a venous valve positioned on the venous line;
an arterial valve positioned on the arterial line;
a first valve positioned on the first line;
a second valve positioned on the second line;
an electronic controller operatively coupled to the blood pump, the venous valve, the arterial valve, the first valve, and the second valve, wherein the electronic controller is configured to:
activate an inflow phase that comprises closing the venous valve and the second valve, opening the arterial valve and the first valve, and operating the blood pump in a forward direction to withdraw blood from the patient through the single needle into the blood circuit and into the accumulator reservoir via the first line; and
activate an outflow phase that comprises opening the venous valve and the second valve, closing the arterial valve and the first valve, and operating the blood pump in the forward direction to move blood from the accumulator reservoir into the blood circuit via the second line and through the dialyzer and back into the patient through the single needle.

14. The system of claim 13, wherein the inflow phase is stopped when the accumulator reservoir is filled with blood.

15. The system of claim 13, wherein the inflow phase is stopped when the blood pump is operated in a forward direction for a preset period of time.

16. The system of claim 13, wherein the electronic controller is further configured to sync an ultrafiltration flow of dialysate through the dialyzer with the outflow phase.

17. The system of claim 13, wherein the blood pump operates at a substantially similar flow rate during the inflow phase and the outflow phase.

18. The system of claim 13, wherein the blood pump operates at a first flow rate during the inflow phase and a second flow rate during the outflow phase.

19. The system of claim 18, wherein the first flow rate is faster than the second flow rate.

20. The system of claim 18, wherein the first flow rate is slower than the second flow rate.

21. A method of retrofitting a dual-needle dialysis system to operate as a single-needle dialysis system, comprising:
forming a Y-junction between a venous line and an arterial line of a blood circuit of the dual-needle dialysis system;
replacing first and second needles of the dual-needle dialysis system with a single needle at the Y-junction;
replacing a saline source of the dual-needle dialysis system with an accumulator reservoir;
inserting the single needle into a patient;
activating an inflow phase of the dialysis system that includes withdrawing blood from the patient through the single needle into the blood circuit of the dialysis system and into the accumulator reservoir of the blood circuit; and
activating an outflow phase of the dialysis system that includes moving blood from the accumulator reservoir through a dialyzer of the dialysis system and back into the patient through the single needle.
